(19) United States
(12) Patent Application Publication
(10) Pub. No.: US 2024/0308139 A1
(43) Pub. Date: Sep. 19, 2024

(54) COMPUTER-IMPLEMENTED METHOD AND SYSTEM FOR PRODUCING AN ORTHOPEDIC DEVICE

(71) Applicant: Ottobock SE & Co. KGaA, Duderstadt (DE)

(72) Inventors: Manuel OPITZ, Munich (DE); Felix GUNDLACK, Herzogenaurach (DE); Max SCHNAUBELT, Munich (DE); Clemens RIETH, Boeblingen (DE)

(21) Appl. No.: 18/612,112

(22) Filed: Mar. 21, 2024

Related U.S. Application Data
(63) Continuation of application No. 16/955,738, filed on Jun. 18, 2020, now abandoned.

(30) Foreign Application Priority Data
Dec. 27, 2017 (DE) 10 2017 131 323.0

(51) Int. Cl. B29C 64/386; A61F 2/50; A61F 2/66; A61F 5/01; B29L 31/00; B33Y 10/00; B33Y 30/00; B33Y 50/00; G16H 10/60; G16H 20/40; G16H 50/50

(52) U.S. Cl. CPC B29C 64/386; A61F 2/5046; A61F 5/01; B33Y 10/00; B33Y 30/00; B33Y 50/00; G16H 10/60; G16H 20/40; G16H 50/50; A61F 2002/5049; A61F 2002/505; A61F 2002/6614; B29L 2031/753

(57) ABSTRACT

The invention relates to a computer-implemented method for producing an orthopedic device. The method includes receiving at least one data set with patient data, processing the patient data in order to create a patient model, using the patient model to determine patient parameters, and generating a virtual representation of the orthopedic device while using the patient parameters and device parameters. The method further includes receiving at least one input from at least one user, modifying at least one of the patient parameters or device parameters on the basis of the input, and physically creating the orthopedic device.

### CROSS-REFERENCE TO RELATED APPLICATIONS

This application is a continuation of U.S. patent application Ser. No. 16/955,738, filed Jun. 18, 2020, which is a U.S. National Phase Patent Application submitted under 35 U.S.C. § 371 of Patent Cooperation Treaty Application Serial No. PCT/EP2018/081579, filed Nov. 16, 2018, and entitled COMPUTER-IMPLEMENTED METHOD AND SYSTEM FOR PRODUCING AN ORTHOPEDIC DEVICE, which application claims priority to German Patent Application Serial No. 10 2017 131 323.0, filed Dec. 27, 2017, and entitled COMPUTERIMPLEMENTIERTES VERFAHREN UND SYSTEM ZUR HERSTELLUNG EINER ORTHOP ADISCHEN VERSORGUNG.

U.S. patent application Ser. No. 16/955,738, Patent Cooperation Treaty Application Serial No. PCT/EP2018/081579, published as WO 2019/129419 A1, and German Patent Application Serial No. 10 2017 131 323.0, are incorporated herein by reference.

### TECHNICAL FIELD

The invention relates to a computer-implemented method for producing an orthopedic device and a corresponding system for producing an orthopedic device.

### BACKGROUND

Orthopedic devices comprise orthoses (corrective and preventive) and prostheses (exoprostheses and endoprostheses). Nowadays, prostheses are usually assembled from modular systems. Different base elements (for example, prosthesis socket, prosthetic foot or hand and prosthetic tube) are constructed, which are screwed together by means of standardized adapters. Around the technical construction, a cosmetic device is usually created, which imitates the natural leg or arm as closely as possible. In this regard, there exists a need to adapt the appearance of the patient aid as well as possible to the natural situation. Nevertheless, the difference from a natural extremity often cannot be disguised. Efforts are therefore made to adapt such patient aids to the individual needs or preferences of the patient or wearer.

An example of such an orthopedic device is known from DE 20 2017 000 442 U1.

Endoprostheses are nowadays also individually adapted to the patient by one or more medical experts. In this regard, the aesthetic matching is naturally less important than the customized form and function. A patterned surface is more likely to be functional, so that the embedding behavior can be improved.

It is known to produce suitable orthopedic devices, in particular, orthoses with computer assistance. The final production of the orthosis, in particular, is now increasingly accompanied by computer-assisted methods (see US 2014/0180185 A1). It is therefore often possible to produce highly individual orthoses wherein the device parameters describing the patient aid include personal preferences and functional details. In the prior art, however, the generation of the model of the orthopedic patient aid often takes account of only very little information provided by the patient and/or the specialist personnel overseeing the production of the orthosis, for example, the certified prosthetist/orthotist, orthopedic surgeon or doctor. There is therefore a need to customize relevant orthoses further and to improve the interaction between the persons and systems involved.

The same applies for preventive orthoses (preventers, protectors). However, these can also be worn for medical reasons to protect against injuries, for example, in contact sports, extreme sport types or motor sports. An aesthetic adaptation (customization), in particular, can be an important criterion in medically non-essential devices when making a (purchase) decision. These preventive orthoses are intended to provide targeted support to the patient or wearer against injury or external forces or overloading during movement. This can be, for example, a customized preventive orthosis following a knee injury, which enables dosed loading of the knee during rehabilitation or during sport. Also conceivable is a customized cervical orthosis as a protector for motorcycle riders or motor racing drivers, which protects them against injuries during crashes or accidents. Further examples are customized protectors for contact sports people such as soccer players, ice hockey players, football and lacrosse players, and many others, in both the amateur and professional fields.

### SUMMARY

Proceeding from this prior art, it is the object of the present invention to provide an improved method for producing an orthopedic device. In particular, possibilities are to be provided to produce high-grade customized devices, preferably in an iterative process. Furthermore, the function and acceptability of corresponding devices are to be improved through the provision of a corresponding method.

The present invention achieves this object by the provision of the computer-implemented method according to claim 13.

In particular, the object is solved by means of a computer-implemented method for producing an orthopedic device, comprising the following steps:
a) receiving at least one data set with patient data;
b) processing the patient data in order to create a patient model;
c) using the patient model to determine patient parameters;
d) generating a (virtual) representation of the orthopedic device, while making use of the patient parameters and device parameters;
e) receiving at least one input from at least one user;
f) modifying at least one of the patient parameters and/or device parameters on the basis of the input;
g) physical creation of the orthopedic device making use of the device parameters, patient parameters and/or a model of the orthopedic device generated on the basis of the patient parameters.

One concept of the present invention therefore lies in the use of raw data as supplied, for example, by a tomograph or an (optical) scanner, in order to generate a patient model.

Thus, an optical scanning method, for example, use of a laser scanner, a laser-supported scanner or a stereoscopic optical scanner with structured light, can be carried out to obtain the raw data. In one embodiment, cameras, in particular stereoscopic optical cameras and/or cameras with only one lens system, are used for digital 3D reconstruction from a series of images. Non-optical scan methods can also be used to obtain the raw data. For this, a CT scan or an MRI scan can be carried out.

Additionally or alternatively, the necessary patient data can also be obtained manually, for example, by means of a measuring tape directly on the patient, or by measuring a patient impression as, for example, a plaster negative. Tactile measuring possibilities such as measuring calipers, rules or automated tactile measuring machines are also conceivable.

This patient model can then be used to derive patient parameters which ultimately are necessary for the optimum functioning of the device.

A further concept of the invention lies in taking account of input by a user, whether input by a certified prosthetist/orthotist or by a patient, in the production of the device. Suitable input can contribute strongly to the improvement of the function and to acceptability of the patient aid.

The physical creation of the orthopedic device can comprise a controlling of at least one production machine, in particular, a 3D printer. Generally, in this technological field, additive methods are preferable and result in patient aids which are extremely stable and have a low weight. According to the invention, however, subtractive methods using, for example, a CNC milling machine can also be utilized. Combined methods in which different printers and/or manufacturing machines are used are also conceivable.

A further concept of the invention lies in the optimization of the production process. This presupposes that both the certified prosthetist/orthotist overseeing the production and the patient concerned are involved as closely as possible in the production process. The present invention thus proposes a visualization of the patient model and/or of the device and/or of a model of the device.

According to the invention however, additionally or alternatively, individual patient parameters and/or device parameters can also be visualized. For this purpose (simple) graphics with labels, where relevant, can be employed. For example, graphically illustrated measuring sheets, as are known in this technical field, can be displayed.

The visualization can be interactive or static. A visualization can take place by means of a 2D or a 3D model. In one embodiment, a print-out of the patient model and/or device model takes place, for example, in 2D on paper or in 3D on a 3D printer.

In one embodiment, this visualization is to take place at an earliest possible time point. In one embodiment, a virtual representation of the patient model is generated, wherein the orthopedic device is represented together with the patient model. A corresponding representation can take place using a web server. Thus, images or even 3D data can be visualized by means of a web browser. In another embodiment, a local program can be installed on the computer of the user (certified prosthetist/orthotist or patient), in order to provide a suitable representation of the patient model and/or the device.

In one embodiment, the visualization can be displayed both to the medical user and/or to the patient. For an increased level of acceptability of the orthopedic device, it is helpful, in particular, if the user makes the visualization of the orthopedic device available to the patient in 3D, for example, in a generally accessible system such as a web browser or in 2D, for example, in a generally readable format such as, for example, a PDF file or a JPG image.

The program or the web browser can also be used to acquire input from the user(s). In one embodiment, the representation of the virtual device is aligned with the patient model. This alignment process can take place in an automated or partially automated fashion. The user of the method can use this representation to derive necessary adaptations to the patient model or the patient parameters. Relevant adaptations can be made directly on the patient model or indirectly on the patient parameters. Furthermore, device parameters can be adapted by the user, wherein the user is supported in his or her selection by the virtual representation.

The patient parameters can comprise parameters that specify a neck circumference, a weight of the patient, one or more angles, e.g. a foot angle, a shoulder width, but also a position of an adapter. The device parameters can also comprise at least one design parameter and/or at least one functional parameter and/or at least one design parameter, for example, a color of the orthopedic patient aid, or a pattern used. The reception, as described, of at least one input from at least one user, can comprise a reception of at least one first input from a first user, for example, from a certified prosthetist/orthotist, wherein in one embodiment, the patient model and/or at least one patient parameter takes place on the basis of the input of the first user. The method according to the invention thus enables an interaction with a first user, in particular, a certified prosthetist/orthotist.

In addition or alternatively, the reception can comprise a reception of at least one second input from at least one second user, for example, a patient, wherein preferably a modification of at least one device parameter takes place on the basis of the input of the second user. Particularly in the configuration in which the first and the second user make input, the method according to the invention can enable the synchronization of this input, so that all the amendment proposals are taken into account.

In one embodiment, the method carries out an authentication of the first and/or second user, so that no unauthorized or unwanted changing of the relevant parameters can take place. Preferably, an authorization database which, for example, assigns a plurality of devices to the first user that this user is permitted to process is implemented. For example, this authorization can be based upon the fact that the first user has originally ordered the devices. In addition or alternatively, the authorization database can specify which parameters, in particular which device parameters, are amendable by the second user. Where the second user is a patient, restrictions can be implemented thereby which prevent the second user from changing functionally relevant parameters. Rather, the second user can be enabled exclusively to amend device parameters such as, for example, inputting an appearance of the patient aid.

In one embodiment, the authorization database can also be used to assign particular authorization levels to particular users of the same type, for example, a certified prosthetist/orthotist or a doctor. Thus, for example, a certified prosthetist/orthotist who has already carried out a plurality of orthopedic devices in accordance with the method described or who is marked as an expert in the system could have access to a plurality of parameters (expert mode).

In one embodiment, the patient data additionally comprises contact data of a second user or the patient. The method can comprise an electronic transmission of a message to the patient prompting the patient to undertake an input or the previously described inputs. In one embodiment, the message contains a URL which enables the user to access a corresponding input mask. Alternatively and additionally, a user recognition and/or a password can be included. Additionally or alternatively, the patient data can be used to authenticate the patient. Preferably, a patient can only make inputs when he or she has been able to authenticate himself or herself successfully.

The object mentioned in the introduction is further achieved with a computer-readable memory store with instructions for implementing one of the methods already described when the instructions are carried out on at least one computer unit.

Similar advantages arise to those described in relation to the method.

Furthermore, the object is achieved with a system for producing an orthopedic device which preferably carries out at least some of the steps described in relation to the method.

In one embodiment, the system is a system having:
a design server, which comprises:
at least one digital interface for receiving a data record with patient data;
at least one database with training data for creating a patient model on the basis of the patient data and the training data;
at least one computer unit for generating 3D data of the orthopedic device; and
a visualization apparatus, in particular a web server, which is configured to display the 3D data in the form of a virtual representation of the device and to receive at least one input from at least one user;
   wherein the computer unit uses the input
a) to modify device parameters and/or patient parameters, and
b) on the basis of the device parameters and the patient parameters, to generate production data for the physical creation of the orthopedic device.
   The system also produces similar or identical advantages to those already described in conjunction with the method.

The invention will now be described in greater detail using several exemplary embodiments which are illustrated in the drawings.

### IN THE DRAWINGS

FIG. 1 individual components of a system for producing an orthopedic device;

FIG. 2 elements of the production server of FIG. 1;

FIG. 3 individual method steps for producing an orthopedic device;

FIG. 4 example of a foot prosthesis obtained according to the invention; and

FIG. 5 visualization according to the invention of the foot prosthesis of FIG. 4.

### DETAILED DESCRIPTION

In the following description, the same reference signs will be used for the same and similarly acting parts.

FIG. 1 shows some of the components that communicate with one another in the course of the production method according to the invention. This involves a CPO computer 10, a patient computer 100, a production server 50 and a design server 20. All of these components can communicate via a network, connected to one another in the described exemplary embodiment via the internet 1.

The CPO computer 10 comprises an optical scanner 12 for acquisition of the surface structure of a patient. This results in raw data (ScanData).

In the described exemplary embodiment, the production server 50 has a 3D printer 52 and thus can produce any desired orthosis, insofar as the necessary data is provided by the design server 20.

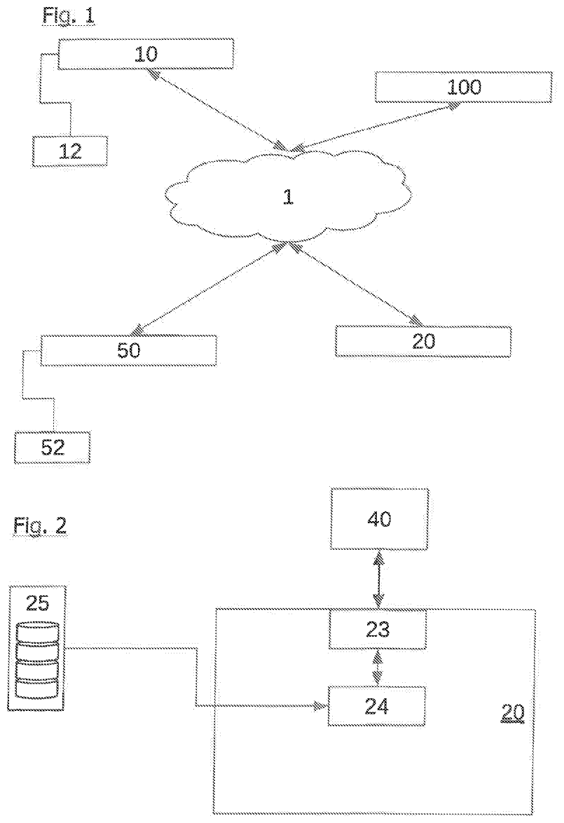

The individual components of the design server 20 will now be described in greater detail by reference to FIG. 2. The design server 20 has a computer unit 24, which at least partially implements the method described below. In addition, a design interface 23 is provided for communication with the already described CPO computer 10 and the patient computer 100. In a preferred exemplary embodiment, this communication takes place via a web server 40, so that the computers 10, 100 do not need any software of their own for communication with the design server 20. Thus, the services provided by the design server 20 can be accessed by means of a web browser.

In addition, a database 25 is provided. This database 25 can supply necessary model data so that the design server 20 can produce models of the orthosis. Furthermore, templates or parameters which enable an individual patient model to be made can be saved in the database 25. The database 25 can further contain authentication information in order to administer access to the design server 20, in particular, to the services offered thereby.

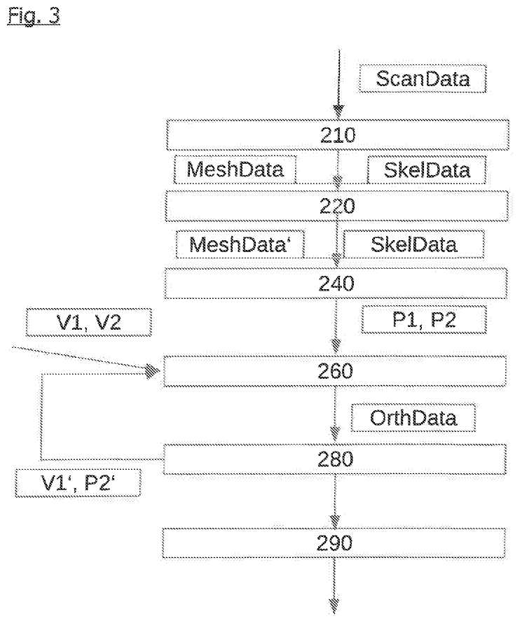

As can be seen from FIG. 3, the design server 20 receives the raw data acquired by the certified prosthetist/orthotist by means of a CPO computer 10. This raw data ScanData can be provided, for example, in the DICOM format. In a pre-processing step 210, a surface network of the patient MeshData and bone data SkelData are obtained from the raw data. The surface network MeshData—Mesh—can be generated as a triangular network, for example, in STL format. In order to obtain the surface network MeshData, surface points are extracted from the raw data ScanData, and the point cloud obtained is embedded in a corresponding network.

In the case of the bone data SkelData, a modelling as a triangular network is also possible according to the invention. Preferably, however, joints and joint connections are modeled, for example, by means of vectors and included in a suitable data structure. Furthermore, for each joint, the usual degrees of freedom with regard to rotational and/or translational movements are stored.

The surface network MeshData and the bone data SkelData can be optimized and validated in a subsequent data validation and optimization step 220. In one exemplary embodiment, a displaying of the data takes place in step 220, wherein corrections are undertaken automatically or computer-assisted. On the basis of the corrections made, a corrected surface network MeshData′ results. The correction can comprise an alignment of the bone data SkelData according to a pre-defined, possibly standardized alignment, wherein the corrected surface network MeshData′ is deformed according to the alignment of the bone data SkelData.

The corrected surface network MeshData′ and the bone data SkelData are processed in a patient parameter extraction step 240. Preferably, in this step 240, with the aid of the database 25, a patient model is obtained. On the basis of this patient model, patient parameters P1, P2 are derived. These patient parameters P1, P2 can be used in the step of orthosis model creation 260 in order to generate a model of the orthosis. Preferably, some device parameters V1, V2 which provide parameters of the orthosis are already available. The orthosis model and possibly also the patient model can be visualized in a visualization step 220.

In one exemplary embodiment, the visualization can be used to adapt some of the parameters, for example, the device parameter V1 and the patient parameter P2. A corresponding adaptation can be carried out by the certified prosthetist/orthotist or, where relevant, by the patient. The adaptation can be performed in one step or in separate steps. After a change of the parameters, in a new orthosis model creation 260, an updated model of the orthosis can be created, for example, using the modified device parameter V1′ and the modified patient parameter P2′. This results in orthosis model data OrthData, which, if met with the approval of the user after a renewed visualization 280, is passed to the production server 50 in order to initiate an orthosis production 290.

In one exemplary embodiment, the data validation and optimization step 220 comprises an alignment correction, for example, according to a particular standardized specification.

In order to correct the position of a scan, the surface network MeshData and the bone data SkelData are needed. Bone data SkelData can be, as described, a simplified bone framework which lies in the interior of the acquired object and thus within the surface network MeshData. In one exemplary embodiment, data which models the interaction between the bone data SkelData and the surface network MeshData is available.

For example, vectors can specify distances or support sites within the surface network. Corresponding vectors can be obtained based upon templates that are stored in the database 25.

A movement of the bones for the alignment correction leads to a deformation of the modeled 3D object and thus to an amended surface network.

According to the invention, the anatomical conditions can be taken into account. In one exemplary embodiment, making use of the template, the bone data SkelData is adapted to the surface network obtained and is improved by means of further process steps in order to be able to model the most realistic possible deformation. The resulting corrected surface network MeshData′ can be used for the extraction of patient parameters.

For example, in the production of a patient-customized ankle-foot orthosis (AFO), a lower leg scan can be examined. According to the invention, in step 220, the scan or the associated raw data ScanData can be brought into a corrected position. For this purpose, in a first step, the orientation of the foot is identified and brought into a defined orientation.

In order to be able to assess the position during the scan, angles of the bone model—bone data SkelData—are investigated together with further biometric axes and planes. If these angles deviate from a selected measurement, the bone data SkelData is adapted/aligned, so that the scan is also changed (corrected surface network MeshData).

According to the invention, the following steps are carried out, for example:
creating a patient model based upon the bone data SkelData and the surface data MeshData;
finding the foot in the patient model;
using the existing data in order to determine a support plane;
modifying a model based upon the bone data SkelData until the support plane is oriented parallel to a virtual base surface, for example, rotation about the ankle (first alignment correction);
modifying the model until the angle of a knee joint takes a pre-determined value (second alignment correction);
modifying the surface data MeshData on the basis of the first and second alignment correction to obtain the corrected surface data MeshData′.

The alignment correction described can enable an extraction of patient data or can significantly improve the result.

In one exemplary embodiment, the patient parameter extraction step 240 follows the scheme below.

For the construction of a patient-customized ankle-foot orthosis (AFO), for example, different length and circumference measurements of the foot and lower leg (patient parameters) are needed. In order to extract these from the raw data ScanData, it is possible to proceed as follows:

The surface data MeshData of the patient is aligned and brought into a reference system from which it can be concluded which part of the scan represents the foot and which part the leg. A simplified foot model is then placed in the surface data MeshData.

This foot model, which is possibly stored in the database 25, is known and can be amended on the basis of its degrees of freedom—e.g. length, scaling, rotation of subcomponents, etc.

In an optimization process, the degrees of freedom of the foot model are adapted to surface data MeshData until the correlation of MeshData and the foot model is optimized (with the smallest possible deviation). In an exemplary embodiment, the process continues accordingly with the Significant Points Model (SPM), which is used for the extraction of the measurements.

The SPM consists of points and planes between which measurements are extracted. The measurement extraction points from the SPM are projected onto the surface data MeshData or the corrected surface data MeshData′. This takes place differently according to the measurement type. Circumference measurements require a sectional plane but length measurements need only points.

The dimensions are extracted between these projected points or along the sectional planes. They can be entered, for example, into a measurement sheet. In one exemplary embodiment, the visualization step 220 comprises the creation of a 3D image of the orthosis. In another exemplary embodiment, for the visualization of patient data, a measurement sheet which is similar or identical to that shown in FIG. 5 is displayed and/or printed out.

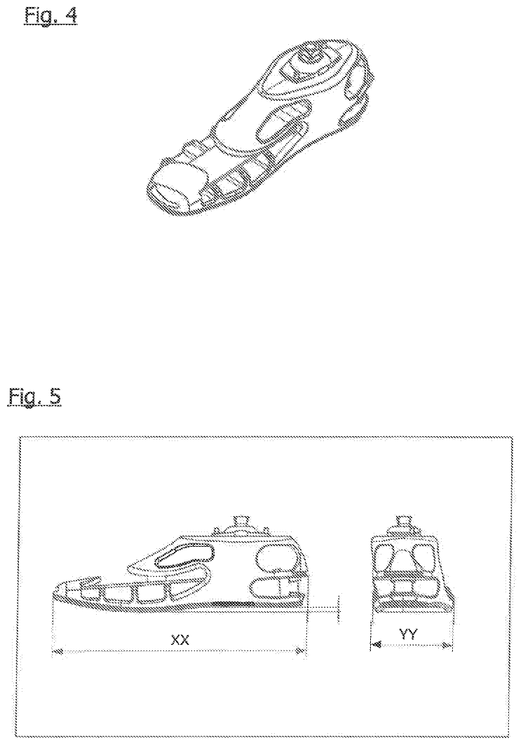

A production method and a system for producing an orthosis have been described above. Using the essential features of the invention, a prosthesis, for example a foot prosthesis as shown in FIG. 4, can also be produced without difficulty.

The production of endoprostheses or preventive orthoses (protectors for rehabilitation and sport) with the same method is also conceivable.

### REFERENCE SIGNS

1 Internet
10 CPO computer
12 Scanner
20 Design server
23 Design server interface
24 Computer unit
25 Database
40 Web server
50 Production server
52 3D printing
100 Patient computer
210 Pre-processing (e.g. bone data extraction)
220 Data validation and optimization
240 Patient parameter extraction
260 Orthosis model creation
280 Visualization
290 Orthosis production
ScanData Raw data
MeshData Surface network
MeshData′ Corrected surface network
OrthData 3D orthosis model data
SkelData Bone data
P1, P2, P2′ Patient parameters
V1, V2, V1′ Orthosis parameters

1. A computer-implemented method for producing an orthopedic device, in particular, an orthosis or prosthesis, comprising:
receiving at least one data set with patient data for a patient;
processing the patient data in order to create a patient model, the processing comprising extracting one or more surface points from the patient data in the form of a point cloud and embedding the point cloud in a surface network representing a surface structure of the patient and in a bone network representing a bone framework that lies within an interior of the surface network;
using the patient model to determine one or more patient parameters;
generating a virtual representation of the orthopedic device while using the one or more patient parameters and one or more predefined device parameters;
receiving at least one input from at least one user;
modifying at least one of the patient parameters and/or device parameters on the basis of the input;
generating a model of the orthopedic device based on the modified patient parameters and/or device parameters; and
physically creating the orthopedic device using the model of the orthopedic device, the physically creating comprising generating and transmitting one or more control signals configured to cause a production machine to produce the orthopedic device.

2. The method according to claim 1, wherein the production machine is a 3D printer.

3. The method according to claim 1, further comprising:
generating a virtual representation of the patient model, wherein the model of the orthopedic device is represented together with the patient model in the virtual representation.

4. The method of claim 1, further comprising:
aligning the virtual representation of the patient model relative to the virtual representation of the orthopedic device.

5. The method of claim 1, wherein:
the patient parameters comprise parameters which specify a neck circumference, or a shoulder width.

6. The method of claim 1, further comprising:
receiving at least one first input from a first user, the first user being a doctor or a certified prosthetist/orthotist, and
modifying at least one of the patient model or at least one patient parameter on the basis of the input of the first user.

7. The method of claim 6, further comprising:
receiving at least one second input from a second user, the second user being the patient, and
modifying at least one device parameter on the basis of the input of the second user.

8. The method of claim 7, further comprising:
authenticating-the first user before receiving the input of the first user and/or
authenticating the second user before receiving the second input of the first user.

9. The method of claim 8, further comprising:
searching an authorization database as to which of the device parameters are amenable by the second user, and
displaying only those device parameters for modification which are amendable by the second user according to the authorization database.

10. The method of claim 1, wherein:
the patient data comprises contact data of the patient, further comprising:
transmitting a message with a uniform resource locator (URL) to the patient making use of the contact data,
authenticating the patient making use of the contact data and/or the patient parameters such that the patient is authorized to provide the input.

11-12. (canceled)

13. The method of claim 1, further comprising producing the orthopedic device with the 3D printer.

14. The method of claim 1, further comprising:
aligning the bone network, the aligning causing a deformation of the surface network.

15. The method of claim 1, wherein the orthopedic device is a foot, and further comprising:
identifying a foot plane in the patient model;
determining a support plane; and
modifying the bone network to orient the support plane parallel to a virtual base surface.

16. The method of claim 1, wherein the orthopedic device is a foot, and further comprising:
identifying a foot plane in the patient model;
determining a support plane; and
modifying the bone network to orient a knee joint to a predetermined value.

17. The method of claim 1, further comprising displaying a virtual representation of the patient model through a web server.

18. The method of claim 1, wherein the device parameters comprise at least one of a material thickness, a flexibility, or a color of the orthopedic device.
